(19) United States
(12) Patent Application Publication
(10) Pub. No.: US 2024/0091512 A1
(43) Pub. Date: Mar. 21, 2024

(54) Adjustable implantable throttle

(71) Applicant: Christoph Miethke GmbH & Co KG, Potsdam (DE)

(72) Inventors: Lucas THIEME, Berlin (DE); Eric SCHLEGEL, Berlin (DE); Christoph MIETHKE, Potsdam (DE)

(73) Assignee: Christoph Miethke GmbH & Co KG, Potsdam (DE)

(21) Appl. No.: 18/038,536

(22) PCT Filed: Dec. 16, 2021
(86) PCT No.: PCT/EP2021/086112

§ 371 Date: May 24, 2023

(30) Foreign Application Priority Data
Dec. 18, 2020 (DE) 10 2020 134 312.4

(51) Int. Cl. A61M 27/00; A61M 39/22

(52) U.S. Cl. CPC A61M 27/006; A61M 39/223; A61M 2202/0014; A61M 2202/04; A61M 2205/04; A61M 2205/3334

(57) ABSTRACT

The invention relates to an adjustable implantable throttle for controlling a drainage rate in implantable drains for cerebrospinal fluid drainage. Despite the existence of proven valves, the invention has set itself the object of improving valves. It solves this by making at least one effective length of at least one channel adjustable.

### CROSS-REFERENCE TO RELATED APPLICATION

This application is a national stage application, filed under 35 U.S.C. § 371, of International Patent Application No. PCT/EP2021/086112, filed on 16 Dec. 2021, which claims the benefit of German Patent Application No. 10 2020 134 312.4, filed 18 Dec. 2020.

### BACKGROUND

Hydrocephalus patients have the following medical problem:

In the skull, the brain is surrounded by a special fluid, the cerebrospinal fluid. This cerebrospinal fluid is constantly produced and reabsorbed to the same extent. In the disease of hydrocephalus, also called hydrocephaly, this balance is disturbed. Since the skull is a closed vessel, enlargement occurs when more cerebrospinal fluid is produced than is reabsorbed. Due to the enlargement, in infants the cranial sutures cannot grow together, and in adults the intracranial pressure increases. Thus, there is an adult hydrocephalus and a child hydrocephalus.

Hydrocephalus can be divided into hydrocephalus internus, hydrocephalus externus, hydrocephalus externus et internus, normal pressure hydrocephalus, and hydrocephalus e vacuo.

Treatment of hydrocephalus was originally performed by merely draining the cerebrospinal fluid. This was done by merely connecting a tube between the skull and a large venous blood vessel or by connecting the skull to the abdomen via a corresponding tube. However, it was soon recognized that the pressure in the skull must have a certain physiological value if other complications should not occur.

Modern therapies for hydrocephalus use an implantable drain, an artificial connection between the cerebral ventricles in the head and a drainage compartment, nowadays usually the abdomen, to set a specific physiologic value.

Various drains are known with which the pressure in the skull of a patient can be treated. The drains are designed to open at a certain critical pressure and release the outflow of liquor, also called cerebrospinal fluid, thus preventing the formation of excess pressure in the skull. Usually, these drains for protecting against excess cerebrospinal fluid (CSF) pressure are called shunts or drains.

The core of the implantable drainage system is an implantable valve that is used to control the drainage. This valve is called hydrocephalus valve. Hydrocephalus valves are regularly implanted just under the skin. Such drains are generally implanted under the skin in the area of the head.

A possible definition of shunts according to Miethke is: any artificial hydraulic connection between a first body part that contains cerebrospinal fluid and a second body part that can receive the same, see Kombogiorgas, D. (2016) “The Cerebrospinal Fluid Shunts” (1st edition), Nova Science Publishers, Incorporated, page 130/131. Concerning hydrocephalus, other sources are the book Normal Pressure Hydrocephalus, Fritsch et al., 2014 Fritsch (2014) and the standards EN ISO 7197 and EN ISO 1463.

All sources contain, among other things, technical terms and definitions concerning hydrocephalus. Furthermore, they contain known active principles and their grouping.

In Kombogiorgas (2016), Miethke proposes a twofold grouping, cf. table 1. In a first subgrouping, he divides valves according to their operating principles into differential pressure valves and hydrostatic valves. In a second subgrouping, he differentiates valves according to clinical functions, into fixed, i.e. non-adjustable, and adjustable valve types.

TABLE 1
Principles of operation of shunts, Kombogiorgas (2016), page 117

| | | |
|---|---|---|
| Valve | Fixed | Adjustable |

Differential pressure valves

| | | |
|---|---|---|
| Silicone slot valves | X | |
| Diaphragm valves | X | |
| Ball cone valves | X | X |

Hydrostatic valve principles

| | | |
|---|---|---|
| Anti Siphon Device Principles | X | |
| Flow reducing principles | X | |
| Gravity-based principles | X | X |

According to Miethke, valves of the hydrostatic valve principle group are defined as valves or valve components whose design objective is to prevent overdrainage (Kombogiorgas (2016), page 67). The aim of the valves in this group is to compensate for the force of hydrostatic pressure acting in the direction of the valve opening (so-called counterbalance).

Valves with hydrostatic operating principle can be differentiated into three types of valves. Their designations are anti-siphon, flow-controlled and gravity-controlled devices. All three valve types have a differential pressure in common. This is calculated as the difference between the downstream pressure minus the upstream pressure (Δp=P_{behind the valve}−P_{before the valve}). The pressure difference which releases a volume flow through the valve is defined as the opening pressure of the valve.

Anti-siphon devices adjust their opening pressure to the level of a suction force acting in the valve. Gravity-controlled devices adjust the opening pressure to their inclination in the earth's gravitational field. Flow-controlled devices, on the other hand, adjust the volume flow passing through them to the pressure difference.

Similar designations for volume flow adapting valves in the prior art are flow rate-dependent, flow regulating or flow reducing valves or devices. Here, the term “flow” is generally equivalent to the term volumetric flow, volume per time.

Each hydrocephalus valve is characterized by a characteristic curve. Alfred Aschoff, MD, describes characteristic curves in “In Vitro Testing of Hydrocephalus Valves”, 1994, page 32. He discusses them therein because shunt valves are flow regulators with unidirectional direction preference. According to Aschoff, they are characterized first by a unidirectional effect, second by an opening and closing characteristic, and third by a specific pressure-flow characteristic. The pressure-flow characteristic is usually nonlinear. According to Aschoff, its course depends on the hydrocephalus valve itself, so that a hydrocephalus valve can only be described by specifying the complete characteristic curve.

Non-adjustable hydrocephalus valves are characterized by one valve characteristic, whereas adjustable valves are characterized by several valve characteristics.

In the case of non-adjustable hydrocephalic valves, it is evident that valves of the group of the hydrostatic valve principle show a certain volume flow, a flow through, depending on the liquor pressure. If an associated volume flow is recorded in a graph for each cerebrospinal fluid pressure, a valve characteristic curve is obtained.

For the adjustable hydrocephalus valves, each setting configures the valve. A different valve characteristic results for each configuration. Practice shows that the different valve characteristics of a valve are similar.

Some significant hydrocephalic valves are described below:

U.S. Pat. No. 8,870,809 B2 (Christoph Miethke GmbH & Co KG) teaches an implantable hydrocephalus system for treating hydrocephalus patients with drugs. The teaching proposes to deliver drugs into the brain ventricles of patients, by means of liquids or fluids or their assistance. For this purpose, the drugs are to be delivered into a void, a cavity of the hydrocephalus system, such that they can be hydraulically pushed from there through a ventricular catheter into the brain ventricles. According to the teachings, this requires a system that in one state receives medicated fluids, and in another state delivers them toward the brain ventricles. The system thus requires a valve and therefore includes a valve arrangement having a valve flap in a housing having an inlet and an outlet. The valve arrangement in the valve opens or closes the inlet of the hydrocephalic system in response to the medicament fluid pressure in the cavity.

According to EP 1 523 635 B1 paragraph (Aesculap AG), DE 38 35 788 A1 teaches a fast-switching ball valve. Phenomenologically, this is an actuating mechanism that moves a ball to release or close a passage opening. When the valve is closed, the ball is pressed against the passage opening by a different pressure of a gas flow. To release the passage order, the actuating mechanism pushes the ball laterally away from the passage opening. In the closed state of the valve, the ball is pressed against the passage opening by an applied pressure, e.g., of a gas flow; to release the passage opening, the actuating mechanism moves the ball away from the passage opening. For this purpose, an actuating element of the actuating mechanism pushes the ball laterally, which then releases from the passage opening or the valve seat of the passage opening. A pulse-driven electromagnet is used as the actuating mechanism for moving the ball, wherein the magnet is pulled back into the starting position by a spring force after actuation.

EP 1 523 635 B1 (Aesculap AG) proposes a valve with compact shape memory alloy actuator. The proposal offers a solution to provide a valve that allows actuation travel in the millimeter range. In principle, the proposal combines a basic body with a passage opening for closing and releasing the passage opening with two wire-shaped elements, in particular shape memory alloy (SMA) wires, as an actuating mechanism. These alternately shorten in response to a change in temperature. The SMA wires are connected to the valve body in such a way that the latter can be moved from a stable position on the passage opening to a stable position next to the passage opening when one element is shortened on one side and back to the stable position on the passage opening when the other element is shortened on one side. In a particularly advantageous embodiment, a valve with binary opening characteristics results. Phenomenologically, a function of a switch results from a position manipulation of a body in front of a passage opening.

US 2015 0182 734 A1 (Christoph Miethke GmbH & Co. KG) discloses an adjustable hydrocephalus valve, programmable gravitational assist, for adjusting pressure in the cranium of a hydrocephalus patient. A diaphragm is used to release a brake to free a rotor to rotate freely about an axis. The resetting of the diaphragm is signaled to a user by an acoustic signal, a click, indicating that the brake has been released or blocked. Since it contains magnets, the rotor can be rotated around its axis by means of a magnetic tool. The rotation is used to set a valve characteristic. The valve has proven itself.

The implantable hydrocephalus valves mentioned above have the following generic features: a housing comprising an inlet, an outlet, and at least one actuating mechanism, wherein the actuating mechanism opens or closes the inlet or the outlet by means of a body in response to cerebrospinal fluid pressure.

These valves have proven their worth.

U.S. Pat. No. 4,676,772 A (Cordis-Cooperation) taught a system for pressure control of cerebrospinal fluid as early as 1985. This comprises an implantable pressure relief valve for fluids, which has a housing and an adjustment unit to adjust the opening pressure of the pressure relief valve. Depending on a pressure applied to the pressure relief valve, a diaphragm is deflected so that a passage between a sealing ring embedded in the diaphragm and a ball is opened. For this purpose, the ball is mounted in a pot with a thread cut in its lateral surface. By means of the thread, the pot can be screwed into or out of a cover, so that a pressure between the ball and the sealing ring can be adjusted. The position of the pot, i.e. the number of screwed-in threads, in the pressure relief valve can be indicated by a magnetic bridge on a display device.

U.S. Pat. No. 4,676,772 A teaches in summary an adjustment of a valve opening pressure, but disadvantageously not the adjustment of a defined volume flow. In addition, the described technique has the disadvantage that setting a valve opening pressure by screwing in a cup can result in plastic deformation of the diaphragm. This occurs when a force is applied to the diaphragm by screwing in the cup too tightly via the ball, the force being beyond the elastic limit of the diaphragm. Precise setting of a valve opening pressure requires precise positioning of the pot in the lid. The pot is rotated in the lid via a magnetic bridge that corresponds to a hand movement of a user. However, the user receives no feedback on the friction or relative position between the pot and the lid. Thus, the pot is not precisely positioned in the lid due to over- or under-rotation by the user, so the valve opening pressure cannot be precisely adjusted.

The so-called Orbis Sigma valve was presented by Sainte-Rose, Hooven, and Hirsch in: A new approach in the treatment of hydrocephalus, Neurosrg, 1987, 66(2), 213-26. The Orbis Sigma valve comprises a sapphire membrane with a bore and a pin piercing this bore. The pin has an undercut in its cross-section in the direction of its end facing the diaphragm. The diaphragm is mounted along its circumference in a housing in a flow channel. The pin is mounted at its end facing away from the diaphragm in the same housing and the same flow channel. If a differential pressure is applied across the diaphragm, it deflects by arching with the pressure gradient. The strength of the curvature and the shape of the undercut in the pin then define a passage. Its size varies with the shape of the undercut. Thus, the Orbis Sigma valve continuously adjusts the size of a passage along a differential pressure applied across a diaphragm in concert with a progression of an undercut.

The disadvantage of the Orbis-Sigma valve is its dependence on the differential pressure. Furthermore, the course of the undercut cannot be assumed to be constant for all patients. Rather, the undercut has to be adapted to the respective severity of the patient's hydrocephalus.

EP 0873761 B1 (DePuy) describes a device for limiting the flow of a liquid. The device shows the principle of a so-called Siphon Guard®. It taught in 1998 a technique to restrict a flow of a fluid from a first area of a patient to a second area. To this end, the device comprises an inlet to receive the fluid from a first area and an outlet to direct the fluid to a second area. Further, the device includes a primary flow path and a secondary flow path, both in fluid communication with the inlet and the outlet. A detector in the device can detect the flow rate, the volumetric flow rate of the fluid so that a decision can be made as to guiding it along the primary or secondary flow path depending on its strength. The detector brings about the decision by comparing a current flow rate with a threshold value. The detector guides the fluid from the inlet to the outlet along the primary flow path when the fluid flow rate is less than a predetermined threshold. Conversely, the detector guides the fluid from the inlet to the outlet along the secondary flow path when the fluid flow rate is greater than a predetermined threshold. The detector is composed of four components, a ball seat, a ball, a leaf spring and a coil spring. The leaf spring pushes the ball out of the ball seat, whereas the coil spring pushes the ball into the ball seat. The difference between the two spring strengths thus defines the threshold value of the detector.

The device for limiting a fluid flow thus digitally adjusted its flow resistance between two states, high flow resistance and low flow resistance. Thus, it has the disadvantage of subjecting the magnitude of a flow resistance between two states to adjustability, but unfortunately not keeping the magnitude of a volume flow constant. Both the size of the passage of the primary flow path and the size of the passage of the secondary flow are predetermined at the factory by the design of the device. Thus, in advancing the prior art, the skilled person is guided to develop technologies to improve the factory sizing of flow paths.

US 2014/0276348A1 (Depuy-Synthes Products, Inc.) from 2013 teaches a surge protection device based on the principle of the so-called “Siphon Guard®”. This comprises a housing with an inlet and an outlet, and a first flow path within the housing. The first flow path connects the inlet to the outlet. In addition, the housing includes a second flow path that also connects the inlet and outlet. Both flow paths have a resistance of flow or flow resistance, respectively. Comparatively, the flow resistance of the second flow path is greater than the flow resistance of the first flow path. Within the first flow path, a valve having a valve seat and a first valve ball and a second valve ball are provided. The first valve ball is movably supported between a closed position, in which the first valve ball is in contact with the valve seat, and an open position, in which the first valve ball is spaced from the valve seat. In this case, the first valve ball is arranged between a second valve ball and the valve seat, and the second valve ball is movably arranged between a closed position and an open position.

Advantageously, the valve opening pressure, the weight force of both balls in relation to the bearing surface of the first ball in the valve seat is adjusted by the position of both balls in the gravitational field of the earth. The greater the angle between a vertical line and the vertical axis of the valve, the lower is the weight force of both balls in relation to the bearing surface of the first ball in the valve seat. Thus, the valve opening pressure decreases with a transition of the valve from a vertical to a horizontal position.

However, matching a valve opening pressure to the valve orientation in the Earth's gravitational field does not correspond to matching a valve opening gap.

The surge protection device also has the disadvantage that the flow resistance of the second flow path is predefined at the factory by its design. The parameters of the flow resistance, such as the number of threads and their thread height, cannot be adjusted after implantation.

EP 1331019 A2 also teaches a flow-controlled device (Codman). This device, self-described in the publication as an anti-siphon shunt, teaches a self-adjusting flow-controlled valve but not an adjustable valve, according to Miethke's differentiation. The anti siphon shunt for regulating a volumetric flow in a patient includes a housing defining a fluid chamber, and an inlet port and an outlet port. The inlet port is for passage of a fluid into the fluid chamber, and the outlet port is for release of the fluid. Additionally, the anti-siphon shunt includes a valve mechanism for regulating fluid flow through the fluid chamber based on the pressure gradient across the fluid chamber. For this purpose, the valve mechanism includes a barrier in the fluid chamber having an opening through which fluid can pass. Furthermore, the anti-siphon shunt comprises a pressure sensor for detecting the external pressure surrounding the fluid chamber and a biasing element, e.g. a spring. The spring is operatively connected to the pressure sensor and is intended to apply a first force against a first surface of a ball. As a result, the ball is pressed against the opening so that passage of the fluid through the barrier is consequently prevented by the fluid chamber. A balancing force acts on a second surface of the ball in the opposite direction of the first force. Both the first and second surfaces are approximately equal in size.

The paper thus teaches the skilled person a technique for closing an opening in a barrier by means of a ball with respect to an opening pressure. The closure is maintained until an opening pressure is reached which exceeds the ratio of the difference between the first force minus the compensation force divided by the cross-sectional area of the opening.

In further embodiment, the document teaches a second technique to move one end of the biasing element, the spring, so that its biasing force changes. For this purpose, the document suggests connecting the peritoneal cavity, also referred to as the abdominal cavity to the fluid chamber through a first channel. This can be, for example, a tube. The proposal further includes a reference chamber, which is also connected to the peritoneal cavity via a second channel. The fluid chamber and the reference chamber are connected by a membrane, the membrane is connected to one end of the biasing element, the spring. Through this connection, the preload of the biasing element is changed as soon as the membrane deflects. The deflection follows the pressure difference between the peritoneal cavity and the reference chamber. The anti-siphon shunt therefore adjusts its opening pressure independently by adjusting the stiffness of a biasing element.

Unfortunately, this document does not teach a way to set a passage, such as a gap between a barrier and a ball, either.

The prior art thus has the disadvantage in common of ignoring the ventricle sizes and their condition. As a result, the state of the art neglects the significance of the drainage volume of cerebrospinal fluid drained from different patients. In physiology, compliance describes the extensibility of body structure. In the field of application of hydrocephalus, this corresponds to the compliance of the ventricles. Since the ventricles vary in geometry and condition depending on the patient, so does their compliance. The compliance of the ventricles is proportional to their volume change and anti-proportional to their pressure change. If compliance is patient-dependent, then as a function of it, the pressure response varies for the same drained volume. The shunts described in the prior art discharge drainage volume in their function as a valve, so they have the disadvantage of a different pressure response depending on the patient.

US 2014 0336 560 (Hakim Carlos) teaches a programmable shunt with a magnetic rotor. The rotor is connected to a cam plate. A tongue of a flexure member rests on the cam plate, so that rotation of the rotor is followed by travel of the tongue along the cam track. Since the cam track has an incline, the tongue is raised or lowered by the rotation. Since the respective height of the tongue preloads a lever that presses a ball into its seat, changing the preload results in an adjusting.

### SUMMARY

An object of the present disclosure is to further improve the treatment of hydrocephalus.

The disclosure so relates to an adjustable implantable throttle for controlling a flow rate in implantable drains for brain water drainage, in particular a hydrocephalus valve, for draining fluid from ventricular systems of patients, the valve comprising at least one housing with a housing interior, at least one first passage for inlet and/or outlet, wherein at least one body is arranged in the housing interior, wherein the body is designed to be movable in at least one direction, and the valve having at least one adjustment unit.

The closest prior art is assumed to be WO 2018/184717 A2 (Christoph Miethke GmbH). This teaches a valve with controllable outflow control of liquor.

For this purpose, it comprises a housing which has an inlet, a passage and an outlet. The passage has a circular profile. A body is mounted in the passage. It is a round body. Since the body diameter is smaller than the diffuser diameter, a gap is formed between them.

Despite the existence of this proven valve, the invention has set itself the task of improving valves. The invention is based on the knowledge that patients react to an outflow of cerebrospinal fluid with varying degrees of well-being. In some cases, the well-being is considerably impaired. This knowledge claims to overcome the disadvantages mentioned above in order to further improve the control of fluid flows from one part of the human body to another.

All modern shunt systems are still subject to a risk of clogging. This clogging, so-called occlusion, requires a complicated cleaning of the clogged shunt, shunt system or one of its parts. Alternatively, the shunt or one of its parts, such as a catheter, hydrocephalus valve, or implanted throttle, must be explanted. Cleaning as well as explanting implies unnecessary surgery, which can be a source of infection. Unnecessary operations block supply capacities and are expensive.

The invention proposes a solution to further improve the risk of clogging of shunts, or hydrocephalic valves, especially implantable throttles.

The improvement is achieved with the features of the main claim. The subclaims describe preferred exemplary embodiments.
[A1] This task is solved in an adjustable implantable throttle for controlling a flow rate in implantable drains for brain water drainage, in that at least one effective length of at least one channel is designed to be adjustable.

With the adjustable implantable throttle according to the invention, or in other words a hydrocephalus valve, the well-being of patients can be surprisingly increased. As a rule, a patient is more insecure if he subjectively realizes that a shunt implanted in him or a hydrocephalus valve, in particular an implantable throttle, could occlude, i.e. clog. Conversely, a patient regularly gains confidence in an implantable throttle when he subjectively realizes that the probability of a throttle blockage is reduced.

Prior art solutions are complicated because they have a plurality of assemblies and a plurality of small parts. In contrast, the throttle according to the invention proposes to minimize the number of small parts. This also minimizes the number of joints. The adjustable implantable throttle according to the invention becomes understandable.

Since the effective length of at least one channel can be adjusted, functioning becomes easy to understand and confidence in the throttle increases.

The setting of an effective length advantageously enables a comparatively precise setting. According to its phenomenon it corresponds to a potentiometer, so that as an advantage a multiplicity of setting states results. Phenomenologically, the possibility of setting many states can be understood as a switching between parallel channels.

Each person's ventricular system varies in size compared to other people. While a first patient has a small volume ventricular system, so-called slit ventricles, a second patient has a wide ventricular system. Since the implantable throttle according to the invention has a variety of setting states, it can be used for a wide range of ventricular system sizes. It can be used for different patient groups because its adjustability creates variability.

If the effective length of the channel is variable and the condition is true that liquor, i.e. cerebrospinal fluid only flows through the effective length then the frictional resistance changes proportionally to the increase or decrease in effective length. Consequently, the outflow velocity decreases with increasing effective length, whereas it increases with decreasing effective length.

In summary, the invention thus enables patient-specific individual adjustment and adjustment of an outflow.

Surprisingly, the proposed solution, setting an effective length, further increases the safety of the valve against occlusion. Since the length is adjustable, the cross-section of the channel can remain constant. Advantageously, it can be designed to be significantly larger than a statistical size, such as the average size of a deposit. This choice of size can prevent deposits or flush out flaking deposits. The proposed solution thus preserves a possible principle for avoiding occlusion: “length before cross-sectional constriction.”
[A2] In a preferred embodiment, the throttle has at least one inlet and one outlet, wherein the inlet and the outlet or the inlet or the outlet each has at least one connection point for an implantable hose system. Advantageously, the connection point is formed as a so-called grommet. A grommet is a tube section turned in one piece, which comprises three segments. The first segment is shaped as a cone, or funnel. The second and third segments are shaped as a cylinder. The radius of the second segment, the middle segment, is smaller than that of the other two segments, so that the second segment forms a taper in the course of the grommet. A tube, in particular a medical catheter, can advantageously be connected in a sealing manner in the second segment by means of a knotted cord, a so-called ligature.
[A3] In a preferred embodiment, the throttle comprises a housing, wherein at least one movable part is arranged in the housing and is configured to be movable from outside the housing.

A movable part, an internal part functions as a switch or as a setting unit. Here, the movement of the part corresponds more advantageously to the switching or setting. The correspondence can be direct or geared. Its advantage is concretized in making a flow rate changeable at constant pressure ratios between inlet and outlet of the throttle.
[A4] If an adjustment disc with at least one bore is provided in the implantable throttle for adjusting the effective length of the channel, so that an adjustment can be made or closed by means of a connection of inlet and outlet depending on a position of the adjustment disc, then, surprisingly, the risk of a blockage, respectively, is further reduced.

The provision of the adjustment disc advantageously establishes a stronger separation of the so-called liquor space from the so-called adjustment space.

The cerebrospinal fluid space can be understood as a space which joins all partial spaces through which the cerebrospinal fluid flows along its passage through a hydrocephalus valve. On the other hand, the adjustment space can be understood as a space which joins all partial spaces which are part of a kinematic chain for the change of state, in particular an adjustment or setting of a valve property.

A blockage usually results from a flow of so-called cerebrospinal fluid through a mechanism or part of shunts, hydrocephalic valves, or implantable throttles. Cerebrospinal fluid is a protein. Its adhesive strength is strong. From this strength follows an increased likelihood of adhesion, pooling, sticking, jamming, i.e., clogging or blocking of the mechanism.

If the hydrocephalus valve according to the invention comprises at least one adjustment disc, for example in the form of a perforated disc, then the cerebrospinal fluid passes exclusively through it to drain through the channel. The channel itself is free of mechanical members, reducing any likelihood of channel obstruction or blockage.

Advantageously, an adjustment of an implantable throttle by a rotation, a turning of an adjusting disc or perforated disc is simple. It is also easy to understand. Therefore, a patient has a chance to understand how the throttle works. In the process of understanding, the patient's chance of recognizing the functional reliability of the throttle increases, so that his confidence in the lasting effect of the throttle grows.

The throttle according to the invention also increases the chance of avoiding surgical cleaning or explantation in the event of an occlusion. If there were a blockage in the channel, this could be bypassed by adjustment. For this purpose, the adjustment disc can advantageously be positioned so that it lies behind the occlusion, i.e. the inflow of cerebrospinal fluid into the channel takes place behind the occlusion. The probability of creating a cerebrospinal fluid outflow even in unfavorable situations is increased. The risk of blockage, i.e. occlusion of the throttle, is also further reduced in the event that the patient is far away from a hospital or a doctor.
[A5] Advantageously, the bore in the adjusting disc is arranged on a circular radius of the disc off center. A small angular rotation can thus advantageously make a noticeable adjustment, since the circle radius corresponds to a transmission ratio.
[A6] The fact that the adjustment disc contains at least one magnet means that it can be rotated by means of a second magnet.
[A7] In a preferred embodiment, at least one spring presses the adjustment disc against the channel. This has the advantage that the adjustable, implantable throttle is secured against unwanted loosening. If the force of the spring is designed to be strong, then a maladjustment of the throttle is made difficult.
[A8] A fluid bridge results from the fact that a bore in the adjustment disc forms a connection that connects at least one space on a first side of the adjustment disc with the channel on a second side of the adjustment disc. Through this fluid bridge, liquor can flow into a passage, in particular a channel. Since the diameter of the bore is larger than an average geometric particle size, in particular a particle diameter of particles in the cerebrospinal fluid, the bore has the advantage of reducing the risk of occlusion. This risk is further minimized because slight twisting of the bore results in shearing, breaking off, or spalling of contaminated, partially occluded, or occluded bore edges. Advantageously, the shearing, or break-off, can be flushed out through the bore and the passage of post-flowing cerebrospinal fluid.
[A9] In a preferred embodiment, the housing wall of the throttle is elastic. This means that it can be advantageously pressed in and used as a switching button or switch.
[A10] Since the adjustment disc is designed to move against the spring force, the adjustment disc can be lifted out of a seat.
[A11] In a particularly preferred embodiment, the throttle has at least two states, a pass state and an adjustment state. In the pass state, the adjustment disc does not touch the channel boundaries. In the adjustment state, on the other hand, the adjustment disc rests on the channel boundaries. The advantage of having at least two states establishes a dual-purpose use. The pass state can be used to clean, flush or test the throttle. The adjustment state can be used to set a desired flow resistance. Since the states are independent of each other, they increase the safety of the throttle.
[A12] The fact that the channel is partially or completely closed in the setting state ensures that cerebrospinal fluid can flow only at intended points, i.e. passages, in particular bores or slits.
[A13] In a preferred embodiment, the channel is open along its length in the pass state. The advantage is that the channel can be cleaned, in particular flushed, as a whole. In a preferred embodiment, a cleaning, or flushing fluid can be introduced for this purpose. Alternatively, pumping can be generated from repeated movement of the embossing to use CSF or a flushing liquid as a flushing agent when the channel is open.
[A14] Since a first state, in particular a pass state, is formed switchable into a second state, in particular an adjustment state, by pressure on the housing, a switching function results. The advantage of this result is further increased by the fact that the switching function integrates a threshold value and uses its integration advantageously. Since the housing integrates a switch with a threshold value function, the throttle according to the invention ensures that it switches exclusively from a pressure at a predetermined level. A threshold value can advantageously be designed according to its minimum height as per standard, it thus reduces the risk of undesired switching of the throttle.
[A15] A particularly advantageous embodiment is one in which the adjustment disc is lifted off in such a way that the frictional forces between the adjustment disc on the one hand and the housing and channel on the other hand, which impede rotation, act only at the center of rotation. This type of design has the advantage that lever arms of only zero to 0.5 mm maximum 2 mm counteract the rotation between the adjusting disc on the one hand and the housing as well as the channel on the other hand. The embodiment thus has the advantage of reducing a braking torque due to frictional forces to a minimum.
[A16] In a preferred embodiment, the adjustment disc can be rotated through 360° in two directions. This arrangement has the advantage that an opening and a closing direction can be designed easily and comprehensibly. In a preferred embodiment, when a hole of an adjustment disc is guided along over a channel, an adjustment, a turning of the adjustment disc in one direction corresponds to a shortening of the effective length of the channel. On the other hand, a rotation in the opposite direction corresponds to an increase of the effective length. Bidirectional twisting thus creates an advantageous prerequisite for setting a flow resistance.
[A17] When the adjustable disc interrupts the connection of inlet and outlet in at least one position, it interrupts an outflow of cerebrospinal fluid. Advantageously, an adjustable effective length thus integrates a switch functionality into the implantable throttle.
[A18] Advantageously, the sealing surface between the disc and the channel is made of an implantable plastic with a hardness of preferably 50 Shore to 80 Shore.

TPE compound materials range from 50 Shore to 90 Shore. Some of them offer the advantage of being approved in accordance with the guideline for the hygienic assessment of organic materials. Approvals are also available in particular for cold or tempered drinking water. Since cerebrospinal fluid has similar properties to water, plastics between 50 Shore and 80 Shore have advantages in normative hygienic assessments for patient safety.
[A19] Since the channel guide runs radially, it creates a prerequisite for simple guiding, i.e. positioning of at least one inlet above the channel. The advantage of simple guiding is concretized in a preferred embodiment. If a rotating body, in particular a perforated disc, is arranged opposite the open channel side, the hole can be guided easily, conveniently, precisely along the channel side. The embodiment is particularly easy to understand by patients due to the advantage of its simplicity. Manufacturing of the channel is simple and inexpensive.
[A20] If the channel cross-sectional area is 0.02 mm² to 0.04 mm² with a radial configuration and a length of 30 mm to 40 mm, the design of the channel cross-sectional area corresponds to the pressure conditions of a patient with hydrocephalus.
[A21] A rectangular duct cross section rectangular is easy to design and manufacture, so it is inexpensive.

Preferred embodiments of the invention are explained by way of example with reference to a drawing.

### BRIEF DESCRIPTION OF THE DRAWINGS

FIG. 1 shows a preferred, first embodiment of the invention in a schematic three-dimensional view.

FIG. 2 shows a preferred, second embodiment in a top view.

FIG. 3 shows a preferred embodiment of the invention in an external view.

FIG. 4 shows a preferred embodiment showing a fluid path.

FIG. 5 shows a preferred embodiment of the inventive throttle in a perspective side view with relaxed diaphragm cover.

FIG. 6 shows a preferred embodiment in two use positions in a side view.

FIG. 7 shows a preferred embodiment of a diffuser as a channel labyrinth.

FIG. 8 shows a preferred embodiment of the invention in a side view.

FIG. 9 shows a preferred embodiment of the invention with a passage in the form of a labyrinth, or with a channel as a labyrinth, or with a labyrinth.

FIG. 10 shows preferred embodiments with respect to different directions for channel geometries.

FIG. 11 shows preferred embodiments with regard to different designs of labyrinths.

FIG. 12 shows a preferred embodiment for the separation of liquor space and adjustment space.

### DETAILED DESCRIPTION

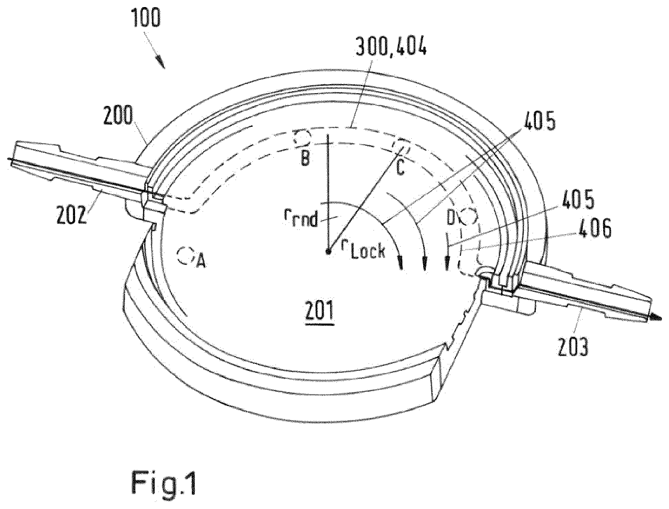

FIG. 1 shows the phenomenon of the hydrocephalus valve 100 according to the invention in its structure in a schematic view from above. In this preferred structure, the hydrocephalus valve 100 comprises five assemblies: a housing 200, a passage 300, with a channel 404, or an internal part (not shown), an adjustment unit (not shown) and a spring element (not shown).

In the preferred design shown, the housing 200 is divided into a housing cover and a housing base, in the space between which, the housing interior 201, an inner part (not shown) is arranged.

In the housing cover and the housing base, passages are made in each of which an inlet 202, and an outlet 203 for liquor are inserted. Together with the housing interior 201 and the channel 404, they act as a fluid connection. In one setting of the preferred design, this allows cerebrospinal fluid to pass from the inlet 202 to the outlet 203 through the channel 404, so that it passes through the adjustable throttle 100, the hydrocephalus valve 100.

In another setting, the passage 300, i.e. in particular the channel 404, is closed by a perforated disc (not shown) acting as an adjusting unit. Its rotation acts to close the channel 404 in the end position, position A, and opens it in the end positions, position B, C or D. Since the different end positions make the effective length 405 of the channel 404, i.e. its effective length flowed through by the cerebrospinal fluid, adjustable, the implantable throttle 100 can thus be described phenomenologically as a linear potentiometer. Advantageously, the potentiometer acts in that a first movement, a rotational movement, releases (not shown) the rotatability of the perforated disc, the rotation being configured to alter a position of the channel inflow.

In a preferred embodiment, the channel 404 is shaped radially, in particular as an arc. Its radius, r_{channel} corresponds to the radius, rₕₒₗₑ of a perforated disc. In alternative embodiments, the channel 404 can have a U-profile or a V-profile in its channel cross section 406.

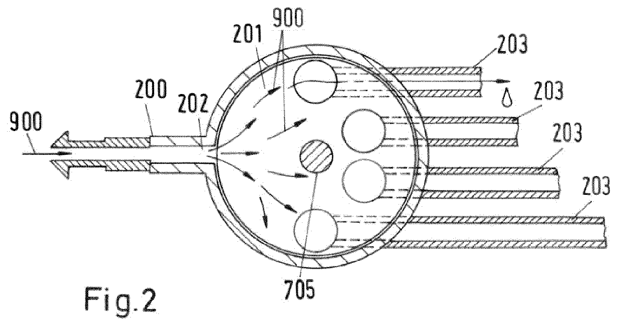

FIG. 2 shows, in broad outline, a preferred embodiment of a hydrocephalus valve 100 in a sectional view from above. It shows that the embodiment is contoured as an interaction of several components in the housing 200. The players are an inlet 202, an internal member (not shown), an adjustment unit (not shown), and a plurality of outlets 203. FIG. 2 shows an exemplary number of four outlets 203. The outlets 203 are arranged along a circle, distributed around a rotor axis 705 in the housing interior 201.

Since the outlets 203 are distributed along a circle, fluid, e.g., cerebrospinal fluid, becomes passable into different outlets 203 when the inner member (not shown) together with the adjustment unit (not shown) opens an outlet 203, i.e., a channel 404 as a result of rotational adjustment of the adjustment unit (not shown).

The respective outlet lengths: L1, L2, L3 and L4 vary. FIG. 2 shows that the outlet lengths for the preferred embodiment can be distinguished. They differ according to the relationship L4>L3>L2>L1.

In alternative embodiments, other relations may apply, e.g., L4>2·L3>3·L2>4·L1, where L1>inlet length. Inlet length to outlet length relations may also apply. Since the outlet lengths vary, the flow resistance varies.

Since the adjustment unit (not shown) is designed as a rotor with one or more holes, fluid 900 passes through one or more holes into one or more outlets 203. Thus, the embodiment corresponds phenomenologically to a potentiometer with different adjustment stages, each adjustment stage corresponding to an outlet 203, or its length L, or the flow resistance.

Thus, in this embodiment, the adjustment unit (not shown) is formed as a symmetrical perforated disc (not shown). The term perforated disc encompasses a variety of disc and disc-like plates or flats. Preferably, the term perforated disc encompasses symmetrical round discs or polygonal discs having at least one perforated disc passage, i.e., a hole, or bore. According to an alternative understanding, neither a perforated disc nor a perforated disc passage need be symmetrical; they may also be asymmetrical. According to this alternative understanding, they have grid profiles, web profiles, or gap profiles with increasing or varying mesh density.

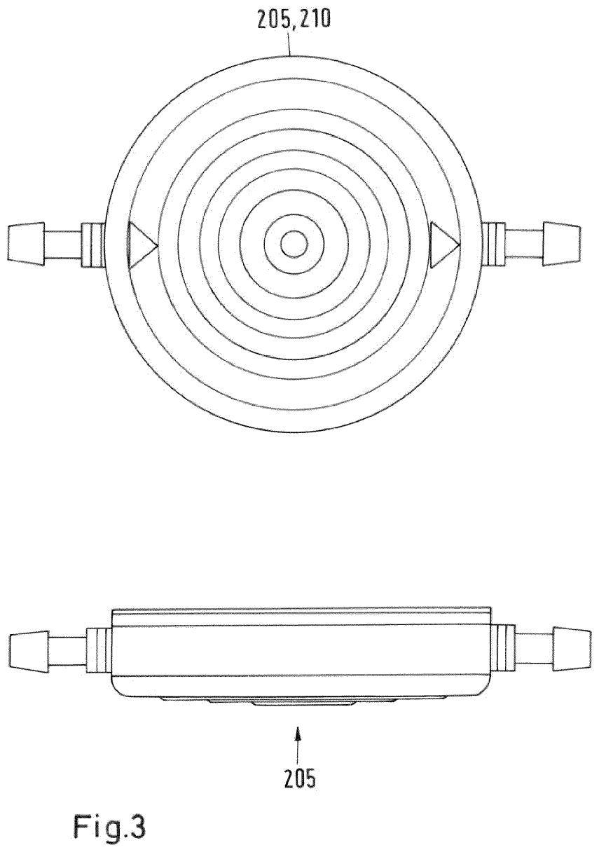

FIG. 3 shows a preferred embodiment of the invention in a view with membrane cover. The membrane cover is an embossing 205, which corresponds in profile section to a staircase with a plurality of steps.

The advantage of embossed diaphragms is a >>click<<. If the diaphragm is deflected from its rest position, and if the deflection overcomes a certain level, then it breaks-down. If the diaphragm breaks-down, it no longer bulges out, but in. Consequently, the tip of the diaphragm rotates 180°. The breakdown occurs quickly and therefore produces a noise that can be heard as a >>click<<.

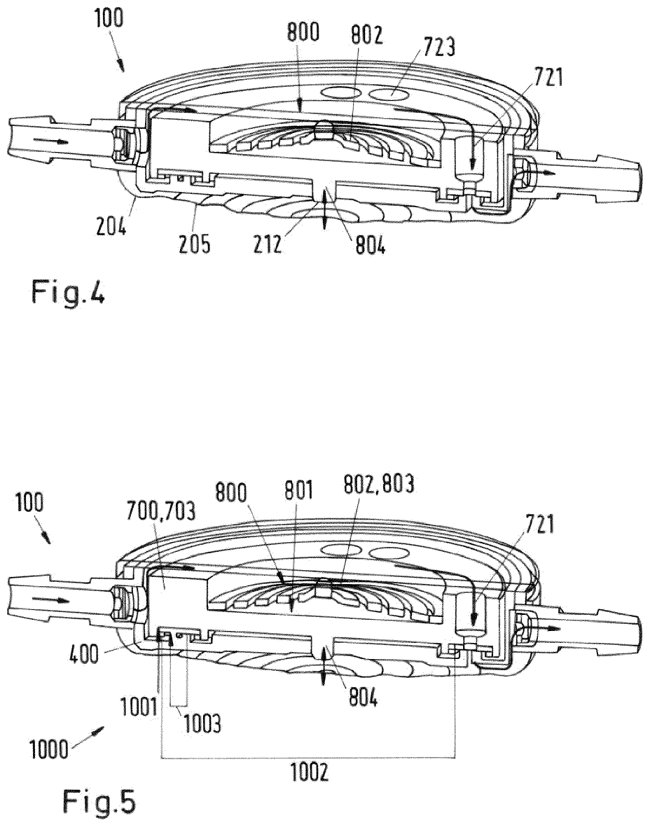

FIG. 4 shows an inventive throttle 100 in a situation where the housing wall has a distance 212 from the pin 804. The distance decreases when pressure is applied to the embossing 205 to release the brake. The pressing deforms the housing cover 204 until it rests on the bolt 804 and further guides it against the spring force of the spring element 800, against a spring 802. The embossing 205 is designed to signal a successful release, i.e., a release of the brake by a click sound, because the upper part of the housing is designed as a click diaphragm, i.e., a stepped round diaphragm (not shown). FIG. 4 further discloses that a fluid (not shown) can flow (arrow) along the inventive throttle 100 when there is no pressure from the embossment 205 acting on the pin 804. In this case, the fluid (not shown) flows through the bore 721.

FIG. 5 discloses a preferred embodiment with brake 1000. Brake 1000 includes braking surfaces 1001 on rotor 703 and inner member braking surfaces 1003 on inner member 400, a silicon 1002, and a spring element 800.

In a preferred embodiment, the spring element 800 comprises a spring seat 801, a spring 802, preferably a coil spring 803, and a pin 804. The spring seat 801 is fixedly formed in the adjustment unit 700. According to the disclosure in FIG. 5, the spring seat is drilled in a rotor 703. In alternative embodiments, it may be milled out, swaged, bolted, clamped, or welded.

By means of the spring element 800, the brake 1000 can be applied or released.

Logically, in a position of use, which can be referred to as the >>brake position<< or >>rest position<<, the Hydrocephalus 100 is secured against opening.

In a preferred embodiment, a brake 1000 is integrated into the adjustable implantable throttle 100 for controlling the flow rate in implantable drains for brain water drainage. It frictionally brakes or releases a movement of an adjustment unit 700, in particular a rotor 703. In the preferred embodiment, the throttle 100 comprises at least one inlet and one outlet, to each of which an implantable tubing system is connected via at least one connection point (not shown). In this case, the implantable throttle 100 comprises a housing, wherein at least one movable part, an adjustment unit 700, is arranged in the housing's housing interior, wherein the part can be moved from outside the housing, preferably by a magnet, so that its movement makes a flow rate variable while the pressure ratios between the inlet and outlet of the throttle 100 remain constant. In this case, the movement of the adjusting unit 700, in particular that of the rotor 703, translates a rotation of an angular input introduced by the magnet into an adjustment of an effective length (cf. FIG. 1, numeral 405) of at least one channel. Alternatively, the adjusting unit 700 can be understood as a rotor 703, an adjusting disc 720 or a perforated disc 720.

In the throttle of the invention for draining fluid from ventricular systems of patients, the translation kinematically results from a release of the brake unit 1000. Since the brake unit 1000 comprises at least one adjustment unit 700 that is configured to move in a first, axial direction of movement, and the axial movement of the adjustment unit 700 is inhibited by the brake unit 1000, a release of the brake 1000 releases the inhibited adjustment unit 700 and creates a stroke that opens a channel inflow.

The brake 1000 secures an adjustment by a frictional connection. A silicone 1002 is provided between the adjusting unit 700 and the inner part 400 to seal. For this purpose, the spiral spring 803 presses the rotor 703 against the housing base 205. This pushing off presses the rotor braking surface 1001 against the silicone 1002, so that the silicone nestles against the inner part braking surface 1003. The valve provides a seal. In an alternative embodiment, a biocompatible plastic or rubber may be used in place of the silicone 1002.

In FIG. 5, the diaphragm cover is relaxed so that no external force is applied to the pin via the diaphragm cover. The force of the spiral spring 803 thus seals a free rotation of the rotor 703, i.e. the perforated disc, by means of kinematics. In the process, the liquor (not shown) flows through the hole 721.

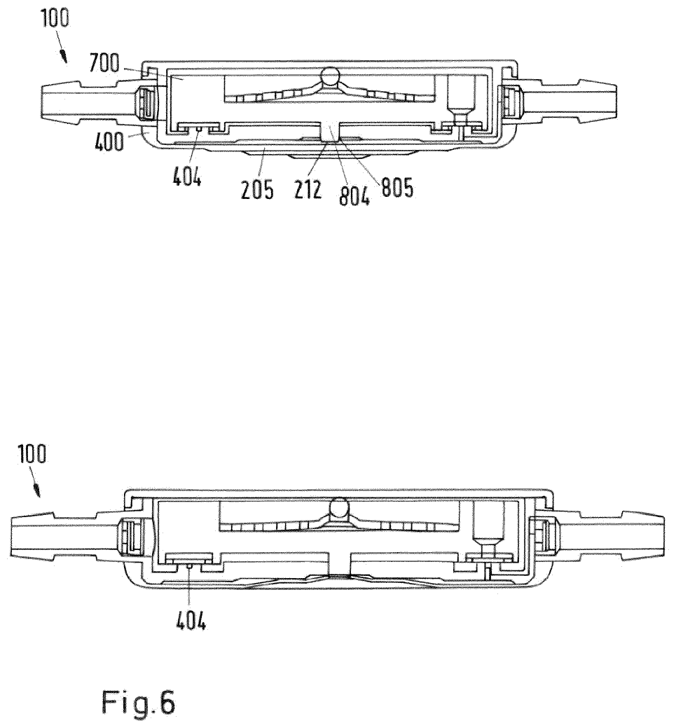

FIG. 6 shows a position of use of the implementable throttle 100 in two states. In the first state, the rest position, the channel 404 is closed. In the second state, the adjustment position, the channel 404 is open.

When the implementable throttle is at rest, a distance 212 is provided between a pin end 805 and the imprint 205.

The figure teaches that in a preferred embodiment, the adjustment unit 700 is mounted in a symmetrical inner part 400. For this purpose, a bolt 804 is integrally formed from the adjusting unit 700, which is held in a bore of the inner part 400.

A depression of the embossing 205 first overcomes the distance 212 before the force of the depression is transmitted to the pin end 805 against the spring. When the force of the indentation, the external force, is stronger than the counterforce of the spring, the adjustment unit 700 is lifted from its seat and the channel 404 is opened.

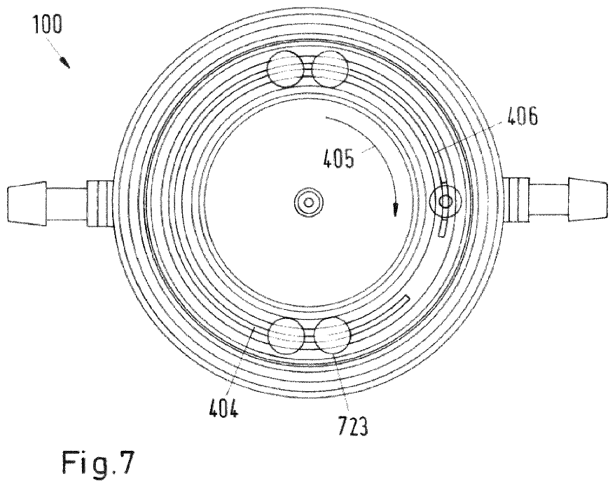

FIG. 7 shows a preferred embodiment of the channel 404 with a U-profile whose course describes an arc of about 340°. In alternative embodiments, the total degree of arc may be 30°, 45°, 60°, 90°, 120°, 160°, 180° or 270°. In alternative embodiments, the degree of arc is in sum more than 20° and less than 200°, or more than 30° and less than 180°.

In this embodiment, the open side length of the channel 404 is at least a quarter of the length of all other closed side lengths of the channel 404, since the three sides of its U-section have the same side length.

FIG. 7 shows an example of the effective length 405 between a bore 721 and the outlet 203. The effective length 405 can be adjusted so that the adjustment unit 700 can be moved by means of a magnetic coupling via at least one magnet 723.

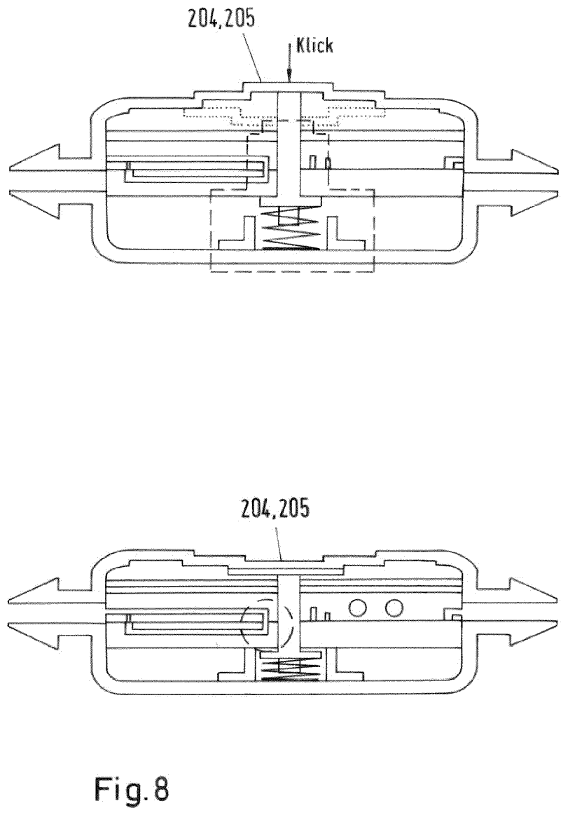

FIG. 8 discloses two switching states of a housing cover 204. The housing cover 204 can also be understood as a housing wall. The inventive throttle is characterized by the fact that an adjustment unit is mounted in the housing cover 204, or a housing wall, which can be moved by pressing-in the embossing 205.

In a first state, the rest position, the diaphragm tip, i.e. the embossing tip, points upward before an indentation and its breakdown, whereas it points downward after an indentation and a breakdown. The punch produces a clicking sound.

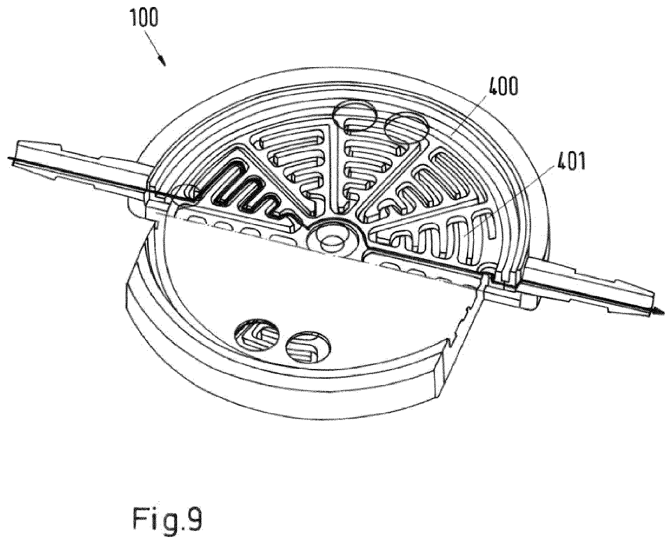

FIG. 9 discloses an inventive throttle 100 with a preferred embodiment of the passage, here, this part, or a part thereof, in particular the channel 404 is formed completely or in parts as a labyrinth 401, in particular milled out of a plate.

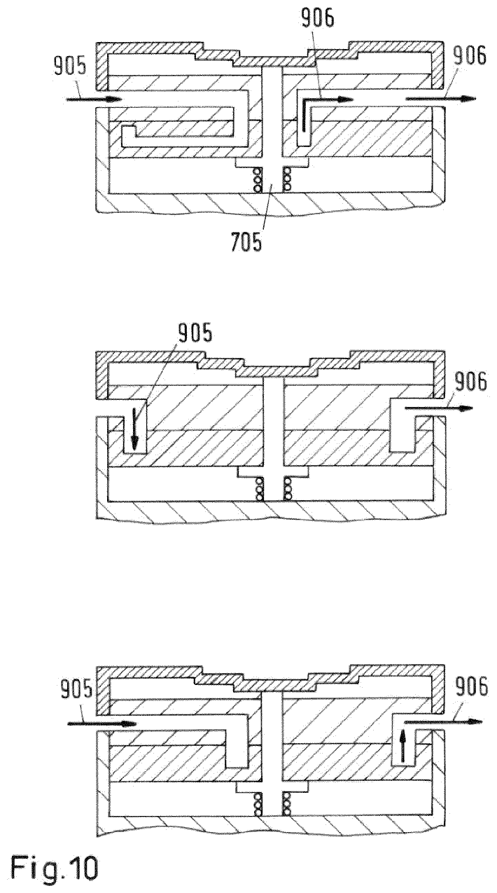

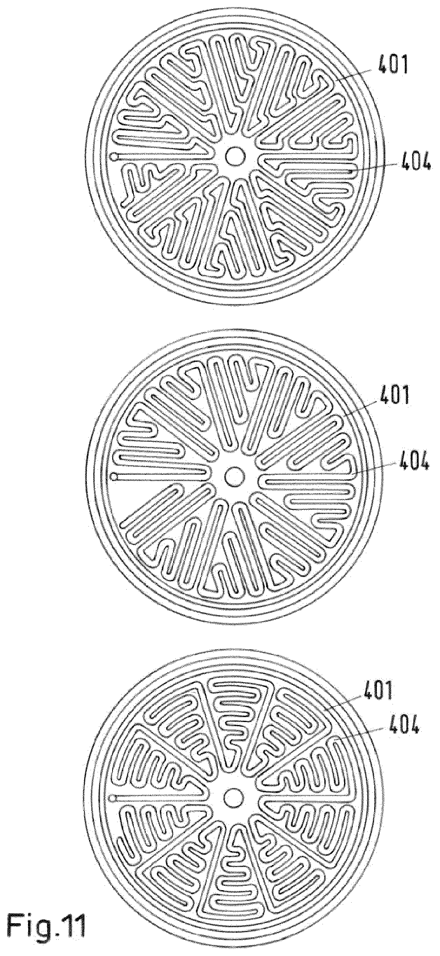

FIG. 10 shows an alternative embodiment of different channelizations of channel shapes, labyrinths 401.

Three preferred embodiments are presented below. They each bear a designation: >>Internal-Internal<<, >>External-External<<, and >>Internal-External<< (not shown). The designations help the reader to classify the embodiments according to one of their functions. It describes a main function of the valve by including two words in each case. The first word describes the location of the labyrinth inlet flow 905, and the second describes the location of the labyrinth outlet flow 906.

For example, an embodiment >>Internal<< describes a hydrocephalic valve 100 with a main function of allowing cerebrospinal fluid 901 to enter the labyrinth 401 close to the axis and to create a fluid bridge 600 in the direction of the hydrocephalic valve center, i.e., its axis 705 between perforated disc 720 and channel 404, i.e., labyrinth 401.

In contrast, an embodiment >>outside-outside<< describes an inventive throttle 100 in which cerebrospinal fluid 901 enters and exits labyrinth 401 spaced apart from the axis.

An embodiment >>inside-outside<< visualizes a teaching to introduce cerebrospinal fluid 901 into the labyrinth 401 close to the axis. Since a thread (not shown) of the labyrinth 401 rises, the cerebrospinal fluid 901 is guided in a spiral whose radius rises. The end of the slope, i.e., the labyrinth exit (not shown) is thus at the outer edge.

FIG. 11 shows alternative designs of a labyrinth, i.e. the routing of channels.

The term labyrinth captures a system of fluid channels, pathways, or trails. Fluid channels vary in their directions.

FIG. 11 discloses that the labyrinth is worked out as a continuous channel whose course follows a shape. In a preferred embodiment, the shape is based on a snail shell. The hydrocephalus valve is thus characterized in that the labyrinth 401 follows a snail profile in its course. A snail-shaped labyrinth course forms a channel, which is long relative to its base area. The profile of the labyrinth may vary so that it may be a U, or a V profile along its length. The hydrocephalus valve of this preferred embodiment may also be segmented, i.e. subdivided into subsections. In this case, each segment, each sub-section has an alternative form of a channel 404 or a labyrinth 401.

In a preferred embodiment, the U-profile has dimensions of 0.4 mm height and 0.4 mm depth. Advantageously, this allows particles with a maximum diameter of 0.03 mm to pass through the labyrinth.

When cerebrospinal fluid enters the valve 100, i.e., the housing 200, it flows through the housing inlet, a grommet, and finally distributes along the surface of the perforated disc 401. When partial volumes of cerebrospinal fluid reach a hole 401, they drain into the labyrinth. Various embodiments are conceivable as to how the fluid passage between a feed channel and the labyrinth can be established.

The inventive hydrocephalus valve described above can be combined with other valves. In this case, the inventive hydrocephalus valve can be arranged upstream or downstream of the other valve in the flow direction/drainage direction. In combination with another valve whose closing body is spring-loaded and opens according to the cerebrospinal fluid pressure, the above-described valve can be used to create a switch effect.

Optionally, a special gravity valve, namely a switchable gravity valve is used in the housing. The gravity valve can be switched off and on. For this purpose, an actuator/switching device is preferably provided in operative connection with the closing part of the gravitation valve.

In an extended embodiment, the inventive hydrocephalus can be electrified. For this purpose, at least one drive is arranged in the inventive hydrocephalus valve, which can rotate the rotor. Furthermore, at least one transmitter and receiver unit and a sensor are to be included. The task of the sensor is to record the so-called intracranial pressure in the patient's head, so that this can be transmitted to an external device by means of the transmitter and receiver unit, if necessary. Conversely, an actuator can receive signals from the external device in order to put an actuator into operation.

The drive is activated by a storable control system in which, for example, a desired time curve of the pressure drop in the cerebrospinal fluid is stored. This curve is compared in the control system with the aid of an algorithm with the pressure values of a pressure measuring device not shown. The difference between the two values results in a control pulse to the electric drive.

The adjustable valve combinations described below, when electronically controlled together with a pressure measurement system not shown, can run a desired pressure curve without any further auxiliary measures. In combination, also in combination with conventional hydrocephalus valves, they can also run at least approximately a desired pressure curve on a purely mechanical basis.

In the embodiment example, magnets are provided in the rotor for the adjustment. Furthermore, magnets are also used in so-called adjustment instruments so that an implanted valve can be adjusted manually by turning the adjustment instruments. Instead of the adjustment device, a storable stepper motor can also be used.

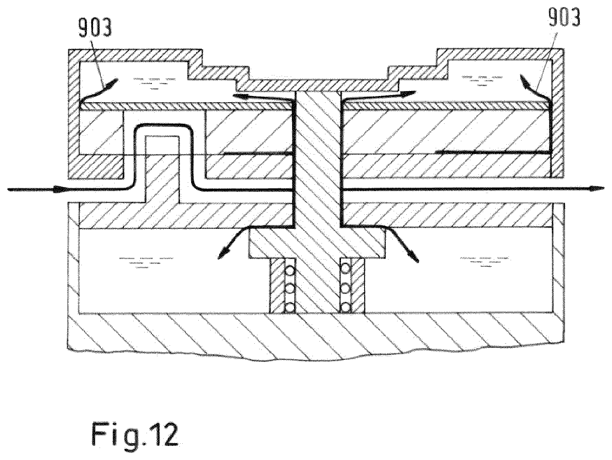

FIG. 12 shows a separation of the liquor space and the adjustment space. Leakage currents 903 are minimized therein.

### LIST OF REFERENCE SIGNS

100 Hydrocephalus valve, adjustable implantable throttle
200 Housing
201 Housing interior
202 Inlet
203 Outlet
204 Housing cover, housing wall
205 Embossing
212 Distance
300 Passage
400 Inner part
401 Labyrinth
404 Channel
405 Effective length
406 Channel cross section
600 Fluid bridge
700 Adjustment unit
703 Rotor
705 Rotor axis
720 Perforated disc, adjusting disc
721 Bore
723 Magnet
800 Spring element
801 Spring seat
802 Spring
803 Coil spring
804 Bolt
805 Bolt end
900 Fluid
901 Liquor
903 Leakage current
905 Labyrinth entry current
906 Labyrinth exit current
1000 Brake unit
1001 Braking surface
1002 Silicone
1003 Inner part braking surface

1.-21. (canceled)

22. An adjustable implantable throttle for controlling a flow rate in implantable drains for brain water drainage, wherein at least one effective length of at least one channel is designed to be adjustable.

23. The adjustable implantable throttle of claim 22, wherein the throttle comprises at least one inlet and one outlet, wherein the inlet and the outlet or the inlet or the outlet each comprises at least one connection point for an implantable tubing system.

24. The adjustable implantable throttle according to claim 23, wherein the throttle comprises a housing, wherein at least one movable part is arranged in the housing, the movable part being designed to be movable from outside the housing, so that its movement makes a flow rate variable at constant pressure ratios between the inlet and the outlet of the throttle.

25. The adjustable implantable throttle according to claim 23, wherein an adjusting disc with at least one bore is provided for adjusting the effective length of the channel, so that an adjustment can be made or closed by means of a connection of the inlet and the outlet depending on a position of the adjusting disc.

26. The adjustable implantable throttle of claim 25, wherein the bore in the adjustment disc is located off-center on a radius of a circle of the adjustment disc.

27. The adjustable implantable throttle according to claim 25, wherein the adjustment disc comprises at least one magnet.

28. The adjustable implantable throttle according to claim 25, wherein at least one spring is provided and the spring presses the adjustment disc against the channel.

29. The adjustable implantable throttle of claim 25, wherein the bore in the adjustment disc forms a connection forming at least one space on a first side of the adjustment disc with the channel on a second side of the adjustment disc.

30. The adjustable implantable throttle according to claim 22, wherein a housing wall of the throttle is elastic.

31. The adjustable implantable throttle according to claim 28, wherein the adjustment disc is designed to be movable against a force of the spring.

32. The adjustable implantable throttle according to claim 25, wherein the throttle has at least two states, a passage state and an adjustment state, wherein in the passage state the adjustment disc does not contact channel boundaries and in the adjustment state the adjustment disc rests on the channel boundaries.

33. The adjustable implantable throttle according to claim 32, wherein in the adjustment state the channel is partially or completely closed.

34. The adjustable implantable throttle according to claim 32, wherein in the pass state the channel is open along its length.

35. The adjustable implantable throttle according to claim 24, wherein a first state can be switched to a second state by applying pressure to the housing.

36. The adjustable implantable throttle according to claim 25,
wherein, when the adjustment disc is lifted off, frictional forces between the adjustment disc, and a housing and channel, which impede rotation, act only in the center of rotation and thus counteract a rotational movement between the adjustment disc and the housing and channel only with a lever arm of zero to 0.5 mm, at most 2 mm, and reduce a braking torque due to the frictional force to a minimum.

37. The adjustable implantable throttle according to claim 25, wherein the adjustment disc is arranged rotatable by 360° in two directions.

38. The adjustable implantable throttle according to claim 25, wherein the adjustment disc interrupts the connection of the inlet and the outlet in at least one position.

39. The adjustable implantable throttle according to claim 25, wherein a sealing surface between the adjustment disc and the channel is realized from an implantable plastic with a hardness of 50 Shore to 80 Shore.

40. The adjustable implantable throttle according to claim 22, wherein the channel extends radially.

41. The adjustable implantable throttle according claim 22, wherein a channel cross-section is an area of 0.02 mm² to 0.04 mm² when arranged radially and having a length of 30 mm to 40 mm.

42. The adjustable implantable throttle according to claim 22, wherein a channel cross-section is rectangular in shape.
